(19) United States
(12) Patent Application Publication
(10) Pub. No.: US 2002/0081264 A1
(43) Pub. Date: Jun. 27, 2002

(54) Expanded perborate salt, use, and method of production

(76) Inventors: Ryan Giffin Moore, Lilburn, GA (US); Hilton G. Dawson, Canton, GA (US); Richard A. DeSenna, Duluth, GA (US); Kenneth Scott Wiley, Oakwood, GA (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 09/858,020

(22) Filed: May 15, 2001

Related U.S. Application Data
(60) Provisional application No. 60/245,540, filed on Nov. 3, 2000.

(51) Int. Cl. A61K 9/46; A61L 9/04

(52) U.S. Cl. 424/44

(57) ABSTRACT

An effervescent compound is disclosed which includes a liquid ingredient, an effervescent system and optionally a solvent. The solvent may be both hydrophilic and have low solubility with effervescent ingredients. The solvent may include an alcohol, a glycol or a glycol ether, for example, but not limited to, 2-butoxyethanol. The effervescent system used in the effervescent compound may be, for example, but not limited to, an expanded perborate salt, anhydrous, or a mixture of an expanded perborate salt, anhydrous, and one or more of sodium bicarbonate, sodium carbonate, potassium bicarbonate and potassium carbonate. The effervescent compound may further include any one, or all, or any combination of the following ingredients: surfactants, bleaching compositions, anti-redeposition agents, binders, lubricants, colors, fragrances, and/or optical brighteners. Further disclosed is a method of producing the expanded perborate salt, anhydrous. Also disclosed is a method for making an effervescent compound, including the steps of providing a solvent, providing an effervescent system in powder form, and mixing the solvent with the effervescent system, thereby producing a free-flowing effervescent compound. The method disclosed may also include the steps of compressing the effervescent compound, and then forming either granules or a tablet.

### CLAIM OF PRIORITY AND CROSS-REFERENCE TO RELATED APPLICATIONS

This application is related to and claims priority to copending U.S. provisional patent application entitled “Expanded Perborate And Method Of Production” filed on Nov. 3, 2000 and accorded Ser. No. 60/245,540, which is entirely incorporated herein by reference.

### TECHNICAL FIELD

The present invention is generally related to effervescent products, and, more particularly, is related to effervescent products including perborate salts, and to a method for producing same.

### BACKGROUND OF THE INVENTION

One major limitation in effervescent cleaning, disinfecting, or other products was the amount and type of liquid and active ingredients that could be incorporated into effervescent formulations. Generally, less than 25% by weight of an effervescent formulation in the form of a tablet or effervescent granules was available for active ingredients. Typically, surfactants, e.g., anionic, nonionic, cationic and amphoteric surfactants, could be used at no more than 5% of the effervescent formulas because these ingredients reduced the storage stability of carbonate-based effervescent products, made conventional granulation or tableting difficult or impossible, and dramatically increased the dissolution time of the resulting products.

Generally, solvents were incompatible with effervescent products. They were either very hydrophobic and were incompatible with aqueous systems or were sufficiently hydrophilic to initiate the effervescent reaction, thus making the product unstable. Many surfactants were available only as liquids containing water or alcohol and could not be employed at any useful level in effervescent formulas. Previously, solvents were primarily used in effervescent tablet production for wet granulation and were evaporated in order to produce a finished tablet.

As noted previously, in typical effervescent tablets, less than 25% by weight of the tablet was usually available for ingredients other than the effervescent system. For example, although up to 75% by weight of the tablet weight could be a binder, only about 10% to 25% was typically used. The effervescent system typically accounted for up to 50% of the tablet. It should be noted that the more of the effervescent system that was used, the quicker the tablet dissolved but less of the tablet was available for other desired ingredients. Lubricants, which help in tablet production, comprised up to approximately 10% by weight of the conventional tablet. Fragrance and color made approximately 2% of the tablet. It was found in conventional tablets that higher levels of fragrance adversely affected tablet stability, dissolution, hardness and tablet production.

One solution has been to replace the typical carbonate-based system with a perborate salt, either anhydrous, monohydrate, or tetrahydrate. The use of sodium perborate anhydrous in non-aqueous liquid built detergent compositions is disclosed in Great Britain Patent Number GB-A-No. 1,205,711 (the '711 patent). The '711 patent, though, gives no further details are given of what is exactly meant by sodium perborate, anhydrous, and does not address improved dispersibility of the detergent in the wash water by addition of sodium perborate anhydrous.

Anhydrous perborate has also been noted as a potential effervescent system, which upon contact with water provides oxygen gas. For example, U.S. Pat. No. 4,772,412 (the '412 patent), issued to Green, et al., discloses a process for making an anhydrous perborate which can be used in liquid detergents. The '412 patent reads that sodium perborate anhydrous as known in the art is unsuitable for use in formulations containing a peroxyacid bleach precursor. The '412 patent further teaches that a mixture of anhydrous perborate and a high level of perborate monohydrate needs to be used in order to provide a more stable effervescent component.

Effervescent compounds have been developed that include anhydrous perborate salts for the purpose of improving the dispensing or dissolution of detergent compositions into the washing water or for improvement of the delivery of the detergent actives of the composition to the wash or for improved sudsing. See, for example, Patent Cooperation Treaty Application Number US99/10008, International Publication Number WO 99/58444 (the 10008 application) and Patent Cooperation Treaty Application Number US99/10007, International Publication Number WO 99/58632 (the 10007 application). However, the 10008 application provides that the anhydrous perborate salt be intimately mixed with a diluent, which could be a process that would add time and cost to the manufacture of the end product detergent. Further, the mixture of the anhydrous perborate salt and diluent is either dehydrated, or the solvent of the non-aqueous diluent is dried, further adding to the time and cost of producing the end product detergent. Additionally, depending on the conditions of the dehydration of the hydrated perborate salt, or the drying process of the diluent, certain amounts of hydrated perborate salt may be present in the product of the 10008 application, which limits the product to uses where bleaching action is accepted.

In addition to the diluent that may be mixed with the anhydrous perborate salt, the 10007 Application provides that the anhydrous perborate salt be coated with a coating material in order to improve its stability. While this relieves the need for incorporation of high levels of perborate, monohydrate, it adds a further step to the production of the detergent compound, thereby increasing time and cost of the manufacture, as well as the weight and size of the end product detergent particles.

One conventional form of sodium perborate anhydrous (oxoborate) is manufactured by and commercially available from Degussa-Hülls AG in Frankfurt, Germany. It can be produced as described in European Patent Number EU0053859, generally by application of heat to a hydrated perborate salt, and under vacuum so as to draw off immediately any vapor released upon the application of heat. The commercially available oxoborate effervesces, but is difficult to use in tablets without the addition of additives. See U.S. Pat. Nos. 4,409,118 and 4,857,224. When loaded with solvents or liquid ingredients, the oxoborate begins to clump when the solvents or liquid ingredients comprise about 60% of its weight. The oxoborate becomes a paste at higher levels of loading with solvents or liquid ingredients.

Thus, a heretofore unaddressed need exists in the industry to address the aforementioned deficiencies and inadequacies.

### SUMMARY OF THE INVENTION

The present invention provides both an effervescent system and a carrier for liquid ingredients to be used in effervescent compounds and a method for making a carrier for liquid ingredients to be used in effervescent compounds. Briefly described, the carrier is an expanded perborate salt anhydrous (EPB) which is both an effervescent material and a carrier which can be loaded with up to 1.2 times its weight with a solvent in which may be dissolved many different materials. The EPB remains a free-flowing dry powder suitable for incorporation into effervescent tablets, granules or powders.

Further, the present invention can also be viewed as providing a method for producing the EPB. The method includes the steps of heating a perborate salt monohydrate at a temperature and for an amount of time to produce the expanded perborate salt, anhydrous.

The present invention can also be viewed as providing for effervescent compounds incorporating EPB, and a method for making the effervescent compounds. In this regard, the method can be broadly summarized by the following steps: providing a liquid ingredient; providing an effervescent system, including EPB, in powder form; and mixing the liquid ingredient with the effervescent system, thereby producing a free-flowing effervescent compound. Alternative embodiments of the present invention include the further steps of dissolving ingredient(s) in a solvent and mixing the resulting solution with the effervescent system thereby producing a free-flowing effervescent compound. Further, the method may include compressing the effervescent compound and forming granules of the effervescent compound. In an alternative embodiment, the method may also include compressing the effervescent compound and forming a tablet from the effervescent compound.

Other methods, features, and advantages of the present invention will be or become apparent to one with skill in the art upon examination of the following detailed description. It is intended that all such additional methods, features, and advantages be included within this description, be within the scope of the present invention, and be protected by the accompanying claims.

### DETAILED DESCRIPTION OF THE PREFERRED EMBODIMENT

The present invention is able to solve the aforementioned deficiencies and inadequacies by using an expanded perborate salt, anhydrous (EPB) in the production of effervescent compounds. The effervescent compounds may be in the form of tablets, granules or powders. EPB is produced by dehydration of a perborate salt, hydrate at elevated temperatures. “Perborate hydrate” in the preferred embodiment of the method of the present invention means a perborate salt, monohydrate.

EPB can adsorb significantly higher levels of liquids, up to 120% of its own weight, and remain free flowing. EPB is surprisingly very easy to tablet, and maintains its flow and tableting characteristics after adsorbing more than its own weight in liquid. Further, the amount of effervescence is sufficient to eliminate the need, if desired, for additional effervescent materials permitting effervescent tablets, granules or powders to be produced that contain up to 60% liquid ingredients.

EPB is produced by heating a perborate salt, hydrate at sufficiently high temperatures for a sufficient time to permit the loss of over 20% of its weight. Thus, EPB is produced by dehydration of a perborate, hydrate at temperatures and time sufficient so as to reduce the weight of the EPB product to less than 80% of the perborate, monohydrate starting material. Generally, the temperature should be, for example, over approximately 120° C. In the preferred embodiment, the temperature of the reaction is approximately 130° C. to approximately 175° C., or higher. While there is not a set upper limit of dehydration temperature, one skilled in the art would understand that at extremely high temperatures, e.g., 600° C., the reaction materials would decompose.

The perborate salt, hydrate is heated generally for approximately 15 minutes to approximately five hours. The reaction of perborate salt, hydrate to form EPB is time-temperature dependent, i.e., the higher the temperature (within reason), the shorter the amount of time it takes for dehydration to occur. For example, at approximately 150° C., the dehydration takes approximately 15 minutes, while at 135° C., the dehydration takes approximately 30 minutes to approximately one hour. In the preferred embodiment of the process of dehydrating the perborate salt, hydrate to EPB, the time for dehydration is fifteen minutes or less, and possibly two to three minutes depending on temperature and other reaction conditions.

The resulting EPB of the present invention has lower residual peroxide levels, lower bulk density, higher porosity and more surface area than commercially available oxoborate. For example, oxoborate has a density of approximately 0.39 g/ml, or, one gram occupies a volume of approximately 2.5 ml. EPB of the present invention, however, has a density of approximately 0.28 g/ml, or, one gram occupies a volume of approximately 3.5 ml.

Without wishing to be bound to any theory, it is believed that the absence of a vacuum in the production of the EPB of the present invention allows some water vapor to re-condense on the perborate salt particles, thereby causing effervescence as oxygen is freed. As the particles effervesce in production, voids or spaces are created in the sodium perborate salt, thereby creating an “expanded” perborate salt, anhydrous, or EPB. It appears that the EPB of the present invention is more of a sodium borate salt than an oxoborate. For example, if commercially available oxoborate is viewed under a microscope, it appears similar to the surface of a loosely packed snowball; however, if EPB of the present invention is viewed under the microscope, the surface of the “snowball” has many craters and voids, and appears much more irregular.

The EPB of the present invention includes a residual amount of peroxide. In the preferred embodiment of EPB of the present invention, the residual amount of peroxide, expressed as hydrogen peroxide, is less than 6% by weight of the composition, and in the preferred embodiment is between approximately 0.5% and approximately 3% by weight of the composition. The hydrogen peroxide of oxoborate, however, is approximately 6% by weight of the oxoborate. Thus, EPB of the present invention can be used in effervescent compositions in which it is not desired or necessary to have a bleaching agent. Further, because it is believed that the peroxide has already been expressed during the production of EPB, as noted above, the result is a product that is lightweight, less dense, and contains more voids that are able to adsorb more liquid active ingredients. Depending upon the desired application, however, it is included as an embodiment of the present invention that oxoborate may be mixed with EPB described herein.

Table 1 below outlines the conditions under which EPB may be produced. EPB may be prepared by heating sodium perborate, monohydrate under ambient pressure. Though the invention is mainly described and explained with respect to sodium perborate, anhydrous, one skilled in the art could envision that other alkali metal perborates, anhydrous might be usable as well.

TABLE 1
Conditions for Production of EPB

| Sample # | Temperature | Time | Weight Loss | H₂O₂ | Density (g/ml) |
|---|---|---|---|---|---|
| 1 | 135° | 0.25 hr | 16% | 2.7% | |
| 2 | | 0.5 hr | 20.5% | 1.7% | |
| 3 | | 1 hr | 21.5% | 0.9% | |
| 4 | | 2 hr | 26.3% | 0.8% | |
| 5 | | 5 hr | 26.9% | 0.5% | 0.28 |
| 6 | 170° | 0.25 hr | 26.9% | 2.7% | |
| 7 | | 0.5 hr | 26.9% | 2.3% | |
| 8 | | 1 hr | 26.9% | 1.5% | |
| 9 | | 2 hr | 27% | 0.8% | |
| 10 | | 5 hr | 27% | 0.8% | 0.27 |

Tableting characteristics of the above samples were compared to commercially available sodium oxoborate. Samples were loaded with 0%, 50% and 100% of their weight with 2-butoxyethanol. The liquid loaded samples were then mixed with 2% sodium benzoate as a lubricant, and two gram tablets were pressed at 0.6 tons using a 19.5 mm punch and die. Table 2 below compares the results of tablets made of conventional oxoborate with tablets made with expanded sodium perborate anhydrous ESPB) of the present invention.

TABLE 2
Hardness Comparison of Oxoborate with ESPB
at Various Loading Levels

| | | Tablet Hardness |
|---|---|---|
| Sample | Loading | (relative factors) |
| Oxoborate - Sample 1 | 0% | 7 |
| | 50% | 3 |
| | 100% | Liquid |
| EPB - Sample 2 | 0% | >40 |
| | 50% | 18 |
| | 100% | 5 |
| EPB - Sample 3 | 0% | >40 |
| | 50% | 20 |
| | 100% | 5 |
| EPB - Sample 4 | 0% | >40 |
| | 50% | 20 |
| | 100% | 6 |
| EPB - Sample 5 | 0% | >40 |
| | 50% | 21 |
| | 100% | 8 |
| EPB - Sample 6 | 0% | >40 |
| | 50% | 23 |
| | 100% | 9 |
| EPB - Sample 7 | 0% | >40 |
| | 50% | 21 |
| | 100% | 10 |

It is apparent from this data that the ESPB of the present invention provides substantially improved loading and tableting characteristics over conventional oxoborate. By using EPB as the carrier to hold all liquid ingredients for the tablets, liquid surfactants may be loaded at high levels into the effervescent products. Solvents, both volatile, e.g., alcohols, ethers; and non-volatile, e.g., glycerine, propylene glycol, can be loaded at high levels and used in the effervescent products. Further, oils and fragrances may be loaded at high levels and used in the effervescent products as well. In the effervescent products of the present invention, solvents can be used to dissolve various active ingredients such as surfactants or quaternary ammonium salts (quats), and loaded onto the carrier, i.e., EPB, for use in the effervescent tablets. It should be noted that this dissolution increases the solubility rate of hydrophobic ingredients. Up to a 1.2:1 loading of liquids to EPB can be used, and the resulting free-flowing powder may be used in effervescent tablets or granules.

The solvents used in the present invention can be used to dissolve many active ingredients which, in addition to being able to increase the amount of active ingredients used in effervescent compounds, also results in an improved solubility rate for the effervescent compound. In various embodiments of the present invention, many different solvents may be used. In the preferred embodiment, the solvent is sufficiently hydrophilic to be used in aqueous products, but does not initiate an effervescent reaction with the effervescent ingredients of the effervescent compound. Preferably, the solvent itself can also be used as an active ingredient in the effervescent compound. In one embodiment of the present invention, the solvent includes a glycol ether. In the preferred embodiment, the solvent comprises, for example, but is not limited to, 2-butoxyethanol. Solvents of this type used in the present invention do not adversely affect either the physical or chemical stability of the effervescent compound, and also provide the possibility of tableting or granulating the effervescent compound.

The effervescent compound including the solvent described above may be formed into a tablet. For the purposes of this document, “tablet” means both tablets and the granular form of effervescent products, and “granular” means generally uniform-sized compressed mixtures. Tablets produced using EPB of the present invention are shelf stable and have good physical properties. For example, but not limited to these physical characteristics, the tablets may be hard; non-friable; of minimum size, both in weight and dimension for the desired application; and have an increased dissolution rate over tablets not made with the EPB of the present invention.

The effervescent system of the present invention may include, for example, but is not limited to, EPB, or EPB plus a mixture of an acid and one or more of sodium bicarbonate, sodium carbonate, potassium carbonate and potassium bicarbonate. Typically, the effervescent system is in a powdered form. If EPB is used as the effervescent system, the liquid ingredients may be present in the effervescent compound of the present invention in an amount up to approximately 50% by weight of the composition. If, however, a carbonate/acid system is used, the liquid ingredients may be used up to an amount of approximately 35% by weight of the effervescent compound. If the solvent is used alone with the effervescent system, and no other ingredients are added, the solvent itself may act as a cleaning ingredient, including, for example, but not limited to, a “degreaser,” which is a compound that will render oils miscible with water.

Many other ingredients, preferably in powder form, may also be added to the solvent and included in the effervescent compound. Desirable, but not necessary, characteristics of these ingredients include being compressible, free flowing, concentrated, and non-sticky. These additional ingredients that may be added to a solvent and then loaded onto the EPB of the present invention include, but are not limited to, any one, all, or any combination of the following: surfactants, bleaching compositions, optical brighteners, anti-redeposition agents, chelating or sequestering agents, binders, lubricants, colors, and/or fragrances. These ingredients may be used in any combination, depending on the application sought for the effervescent compound of the present invention. If EPB is used as the effervescent system, these combined ingredients may be present in an amount up to 50% by weight of the effervescent compound. If, however, the carbonate/acid effervescent system is used, these ingredients may be present in an amount up to approximately 65% by weight of the effervescent compound.

Exemplary surfactants that may be added to the effervescent compound include, but are not limited to: synthetic anionic surfactants, which are generally water-soluble alkali metal salts of organic sulfates and sulfonates; non-ionic surfactants, which are generally the reaction products of alkylene oxide with alkyl phenol, or primary or secondary alcohols, or are amine oxides, phosphine oxides or dialkyl sulphoxides; amphoteric; or zwitterionic surfactants and/or soaps. Examples of the bleaching composition that may be used include, but are not limited to, chlorinated isocyanurates, perborate hydrates, persulfates or percarbonates. Examples of anti-redeposition agents include, but are not limited to, acrylates and cellulose derivatives. The binder used in the effervescent of the present invention may include, for example, but are not limited to any one, all, or any combination of the following: starch and starch derivatives, cellulose and cellulose derivatives, carbohydrate gums, sugars, resins, proteins and inorganic salts. Examples of lubricants that may be used in the effervescent compound of the present invention include, but are not limited to any one, all, or any combination of the following: sodium benzoate, sodium stearate, magnesium stearate, aluminum stearate, stearic acid, mineral oil and polyethylene glycol.

There are several examples of effervescent products that may be made with the carrier of the present invention, for example, including, but not limited to, a carpet cleaner, a dish detergent, a glass cleaner, a laundry detergent and/or a hard surface cleaner/disinfectant. The effervescent carpet cleaner that may be made using EPB of the present invention can include up to 18% by weight of the composition of solvent, which had not previously been accomplished in previous carpet cleaners. Further, the carpet cleaner using EPB of the present invention can include surfactant(s) up to 15% by weight of the composition, which is three times the usual concentration of surfactant that has been used heretofore in effervescent cleaners. Fragrance may be included up to approximately 3% by weight of the composition. Further, the carpet cleaner may include liquid ingredients up to 36% by weight of the effervescent cleaner, which is five to ten times the amount that was previously possible for the liquid concentration in conventional cleaners. Further, the carpet cleaner tablet made from EPB of the present invention has a dissolution time of less than five minutes at 40° C. when dissolved for use as a cleaning aid.

Another example of a cleaner that may be produced using EPB of the present invention is a glass cleaner. The glass cleaner of the present invention may be made in, for example, but not limited to, tablet form. The glass cleaner may incorporate up to 30% solvent by weight of the composition, which had not previously been accomplished in conventional effervescent products. Further, the glass cleaner may include up to 5% by weight surfactant(s), and liquid ingredients up to 35% by weight of the composition. Further, the glass cleaner also has a dissolution time of less than approximately five minutes at approximately 40° C. when dissolved for use as a cleaning aid.

Another example of a possible cleaner that may be produced using EPB of the present invention is a laundry detergent. This laundry detergent of the present invention may include liquid surfactant(s) in an amount up to 24% by weight of the composition, an amount that had not previously been accomplished in effervescent cleaners. The laundry detergent may also include up to 5% fragrance, which is two to five times the amount that has been used in typical effervescent formulas. The laundry detergent has a dissolution time of less than five minutes at 20° C. when dissolved for use as a cleaning aid.

Another example of a possible cleaner that may be produced using EPB of the present invention is a hard surface cleaner/disinfectant. The hard surface cleaner of the present invention may include solvent in an amount up to 24% by weight of the composition, an amount that had not previously been accomplished in conventional hard surface cleaners. The hard surface cleaner may also include up to 15% quats, which is five times the amount that has been used in conventional effervescent tablets and is important to rendering the tablet more effective, or for making the tablets smaller. The hard surface cleaner may include surfactant(s) in an amount up to 5% by weight of the composition, and active ingredient(s) in an amount of approximately 39% by weight of the composition. The hard surface cleaner also has a dissolution time of less than approximately five minutes at approximately 40° C. when dissolved for use as a cleaning aid.

Also included within the scope of the present invention is a method for making an effervescent compound that includes the steps of providing a solvent and/or liquid ingredient, providing an effervescent system in powder form that includes EPB, and mixing the solvent with the effervescent system, thereby producing a free-flowing effervescent compound. The method may also include the steps of compressing the compound and forming either granules or a tablet of the effervescent compound. The solvent used in the step of providing a solvent is preferably both hydrophilic and has low solubility with effervescent ingredients. The solvent may be, for example, but is not limited to, any one or any combination of the following: glycols, alcohols and glycol ethers, e.g., 2-butoxyethanol.

The effervescent system that is used in the step of providing an effervescent system in powder form may be, for example, but is not limited to EPB and/or a mixture of an acid and one or more of sodium bicarbonate, sodium carbonate, potassium carbonate and potassium bicarbonate, or a mixture of EPB with the acid/carbonate-based system. The method may further include the steps of providing other ingredients in a powder form that are to be mixed with the solvent and the effervescent system, thereby producing the effervescent compound. These ingredients include, but are not limited to, any one, all, or any combination of the following: surfactants, bleaching compositions, optical brighteners, anti-redeposition agents, binders, lubricants, colors, and/or fragrances. If necessary, these ingredients may be mixed with or dissolved in a solvent or mixture of solvents, and, if necessary, distilled to remove any water or low boiling-point alcohol before being loaded onto the EPB of the present invention. The resulting liquid can then be used in the effervescent compound. It should be noted that the resulting effervescent compound including the EPB of the present invention is stable and has good tableting and dissolution characteristics.

It should be emphasized that the above-described embodiments of the present invention, particularly, any “preferred” embodiments, are merely possible examples of implementations, and are merely set forth for a clear understanding of the principles of the invention. Many variations and modifications may be made to the above-described embodiment(s) of the invention without departing substantially from the spirit and principles of the invention. All such modifications and variations are intended to be included herein within the scope of this disclosure and the present invention and protected by the following claims.

Therefore, having thus described the invention, at least the following is claimed:

1. A composition for use in production of effervescent compounds comprising:
an expanded perborate salt, anhydrous.

2. The composition of claim 1, wherein the expanded perborate salt anhydrous, adsorbs a high percentage of liquid active ingredients.

3. The composition of claim 1, wherein the expanded perborate salt, anhydrous adsorbs liquid active ingredients that comprise approximately 50 percent by weight of the composition.

4. The composition of claim 1, wherein the expanded perborate salt anhydrous is an expanded alkali metal perborate, anhydrous.

5. The composition of claim 4, wherein the expanded alkaline earth metal perborate anhydrous is expanded sodium perborate, anhydrous.

6. The composition of claim 1, wherein the expanded perborate salt, anhydrous has a residual amount of peroxide.

7. The composition of claim 6, wherein the residual amount of peroxide is between approximately 0.5% and approximately 3% expressed as hydrogen peroxide.

8. The composition of claim 1, wherein the composition further comprises oxoborate.

9. An expanded perborate salt anhydrous compound, prepared by a process comprising the steps of:
providing a perborate salt, monohydrate; and
dehydrating the perborate salt, monohydrate under ambient pressure.

10. The product by the process of claim 9, wherein the step of dehydrating the perborate salt, monohydrate comprises the step of heating the perborate salt, monohydrate.

11. The product by the process of claim 10, wherein the step of heating the perborate salt, monohydrate comprises the step of placing the perborate salt, monohydrate in an oven.

12. The product by the process of claim 11, wherein the step of placing the perborate salt, monohydrate in an oven comprises placing the perborate salt, monohydrate in a dry oven.

13. The product by the process of claim 10, wherein step of heating the perborate salt, monohydrate comprises heating the perborate salt, monohydrate at a temperature in the range between approximately 130° C. and approximately 175° C.

14. The product by the process of claim 10, wherein step of heating the perborate salt, monohydrate comprises heating the perborate salt, monohydrate for approximately fifteen minutes to approximately five hours.

15. The product by the process of claim 9, wherein the step of heating the perborate salt, monohydrate comprises heating the perborate salt, monohydrate for less than fifteen minutes.

16. The product by the process of claim 9, wherein the expanded perborate salt, anhydrous is expanded sodium perborate, anhydrous.

17. The product by the process of claim 9, further including oxoborate.

18. The product by the process of claim 9, wherein the step of dehydrating the perborate salt, monohydrate comprises the step of dehydrating the perborate salt, monohydrate at temperatures and time sufficient reduce the weight of the expanded perborate salt, anhydrous compound to less than 80% of the perborate salt, monohydrate.

19. A method for making an effervescent compound comprising the steps of:
providing a solvent that is both hydrophilic and has low solubility with effervescent ingredients in an amount up to approximately 50% by weight of the compound;
providing an effervescent system in powder form that includes expanded perborate salt, anhydrous; and
mixing the solvent with the effervescent system, thereby producing a free-flowing effervescent compound.

20. The method of claim 19, further comprising the steps of:
compressing the effervescent compound; and
forming granules of the effervescent compound.

21. The method of claim 19, further comprising the steps of:
compressing the effervescent compound; and
forming a tablet from the effervescent compound.

22. The method of claim 19, wherein the step of providing an effervescent system that includes expanded perborate salt, anhydrous comprises providing an effervescent system in powder form that further includes an acid and one or more of sodium bicarbonate; sodium carbonate; potassium bicarbonate and potassium carbonate.

23. The method of claim 19, wherein the step of providing an effervescent system that includes expanded perborate salt, anhydrous comprises providing an effervescent system that includes expanded alkali metal perborate, anhydrous.

24. The method of claim 23, wherein the step of providing an effervescent system that includes expanded alkali metal perborate, anhydrous includes providing expanded sodium perborate, anhydrous.

25. The method of claim 19, wherein the step of providing an effervescent system that includes expanded perborate salt, anhydrous comprises providing an effervescent system that includes an expanded perborate salt, anhydrous that adsorbs liquid active ingredients that comprise approximately 50% by weight of the composition.

26. The method of claim 19, wherein the step of providing an effervescent system that includes expanded perborate salt, anhydrous comprises providing an effervescent system that includes a mixture of an expended perborate salt, anhydrous and oxoborate.

27. An effervescent compound comprising:
a solvent that is both hydrophilic and has low solubility with effervescent ingredients and comprises up to approximately 50% by weight of the compound; and
an effervescent system that comprises expanded perborate salt, anhydrous.

28. The compound of claim 27, wherein the expanded perborate salt anhydrous, adsorbs a high percentage of liquid active ingredients.

29. The compound of claim 27, wherein the expanded perborate salt, anhydrous adsorbs liquid active ingredients that comprise approximately 50 percent by weight of the composition.

30. The compound of claim 27, wherein the expanded perborate salt, anhydrous is an expanded alkali metal perborate, anhydrous.

31. The compound of claim 30, wherein the expanded alkali earth metal perborate, anhydrous is expanded sodium perborate, anhydrous.

32. The compound of claim 27, wherein the expanded perborate salt, anhydrous has a residual amount of peroxide.

33. The compound of claim 32, wherein the residual amount of peroxide is between approximately 0.5% and approximately 3% expressed as hydrogen peroxide.

34. The compound of claim 27, wherein the effervescent system further comprises oxoborate.
